(19) United States
(12) Patent Application Publication
(10) Pub. No.: US 2003/0207274 A1
(43) Pub. Date: Nov. 6, 2003

(54) Human NjmuR1-related gene variant associated with lung cancers

(76) Inventor: Ken-Shwo Dai, Hsinchu (TW)

Correspondence Address: Ladas & Parry, 26 West 61 Street, New York, NY, 10023, US

(21) Appl. No.: 10/102,547

(22) Filed: Mar. 20, 2002

(51) Int. Cl. C12Q 1/68; G01N 33/574; C07H 21/04; C12N 9/00; C12P 21/02; C12N 5/06

(52) U.S. Cl. 435/6; 435/7.23; 435/69.1; 435/183; 435/320.1; 435/325; 536/23.2

(57) ABSTRACT

The invention relates to the nucleic acid and polypeptide of a novel human NjmuR1-related gene variant.

The invention also relates to the process for producing the polypeptide of the variant.

The invention further relates to the use of the nucleic acid and polypeptide of the gene variant in diagnosing diseases, in particular, lung, e.g. small cell lung cancer.

### FIELD OF THE INVENTION

The invention relates to the nucleic acid of a novel human NjmuR1-related gene variant, the polypeptide encoded thereby, the preparation process thereof, and the uses of the same in diagnosing diseases associated with the deficiency of NjmuR1gene, in particular, lung cancers, e.g. small cell lung cancer (SCLC).

### BACKGROUND OF THE INVENTION

Lung cancer is one of the major causers of cancer-related deaths in the world. There are two primary types of lung cancers: small cell lung cancer and non-small cell lung cancer (NSCLC) (Carney, (1992a) Curr. Opin. Oncol. 4:292-8). Small cell lung cancer accounts for approximately 25% of lung cancer and spreads aggressively (Smyth et al. (1986) Q J Med. 61: 969-76; Carney, (1992b) Lancet 339: 843-6). Non-small cell lung cancer represents the majority (about 75%) of lung cancer and is further divided into three main subtypes: squamous cell carcinoma, adenocarcinoma, and large cell carcinoma (Ihde and Minna, (1991) Cancer 15: 105-54). In recent years, much progress has been made toward understanding the molecular and cellular biology of lung cancers. Many important contributions have been made by the identification of several key genetic factors associated with lung cancers. However, the treatments of lung cancers still mainly depend on surgery, chemotherapy, and radiotherapy. This is because the molecular mechanisms underlying the pathogenesis of lung cancers remain largely unclear.

A recent hypothesis suggested that lung cancer is caused by genetic mutations of at least 10 to 20 genes (Sethi, (1997) BMJ. 314: 652-655). Therefore, future strategies for the prevention and treatment of lung cancers will be focused on the elucidation of these genetic substrates, in particular, the genes localized on chromosome 17, a region shown to be associated with the development of lung cancer (Sameshima et al. (1990) Biochem Biophys Res Commun 173:697-703; Kato et al. (1993) Jpn J Cancer Res 84:355-9; Shimizu et al. (1993) Cancer 71:725-8; Levin et al. (1995) Genes Chromosomes Cancer 13:175-85; Abujiang et al. (1998) Oncogene 17:3029-33; Konishi et al. (1998) Oncogene 17:2095-100). Recently, NjmuR1(a novel human protein kinase related to spermatogenesis) transcript mapped on this region (GenBank Accession No. AF305686) raised a possibility that this novel gene may have a role in the tumorigenic process of lung cancer. Therefore, the discovery of gene variants of NjmuR1may be important targets for diagnostic markers of lung cancer.

### SUMMARY OF THE INVENTION

The present invention provides an NjmuR1-related gene variant and the polypeptide encoded thereby as well as the fragments thereof. The nucleotide sequence of the gene variant and the polypeptide encoded thereby can be used for the diagnosis of diseases associated with the deficiency of NjmuR1gene, in particular, lung cancers, e.g. SCLC.

The invention further provides an expression vector and host cell for expressing the polypeptide of the invention.

The invention further provides a method for producing the polypeptide of the invention.

The invention further provides an antibody specifically binding to the polypeptide of the invention.

The invention also provides methods for diagnosing diseases associated with the deficiency of NjmuR1 gene in a mammal, in particular, lung cancers, e.g. SCLC.

### BRIEF DESCRIPTION OF THE DRAWINGS

FIG. 1 shows the nucleic acid sequence (SEQ ID NO: 1) and amino acid sequence (SEQ ID NO: 2) of NjmuR1V.

FIG. 2 shows the nucleotide sequence alignment between the human NjmuR1gene and NjmuR1V.

FIG. 3 shows the amino acid sequence alignment between the human NjmuR1protein and NjmuR1V.

### DETAILED DESCRIPTION OF THE INVENTION

According to the present invention, all technical and scientific terms used have the same meanings as commonly understood by persons skilled in the art.

The term “antibody” used herein denotes intact molecules (a polypeptide or group of polypeptides) as well as fragments thereof, such as Fab, R(ab′)₂, and Fv fragments, which are capable of binding the epitopic determinutesant. Antibodies are produced by specialized B cells after stimulation by an antigen. Structurally, antibody consists of four subunits including two heavy chains and two light chains. The internal surface shape and charge distribution of the antibody binding domain is complementary to the features of an antigen. Thus, antibody can specifically act against the antigen in an immune response.

The term “base pair (bp)” used herein denotes nucleotides composed of a purine on one strand of DNA which can be hydrogen bonded to a pyrimidine on the other strand. Thymine (or uracil) and adenine residues are linked by two hydrogen bonds. Cytosine and guanine residues are linked by three hydrogen bonds.

The term “Basic Local Alignment Search Tool (BLAST; Altschul et al., (1997) Nucleic Acids Res. 25: 3389-3402)” used herein denotes programs for evaluation of homologies between a query sequence (amino or nucleic acid) and a test sequence as described by Altschul et al. (Nucleic Acids Res. 25: 3389-3402, 1997). Specific BLAST programs are described as follows:

(1) BLASTN compares a nucleotide query sequence against a nucleotide sequence database;

(2) BLASTP compares an amino acid query sequence against a =;:7 protein sequence database;

(3) BLASTX compares the six-frame conceptual translation products of a query nucleotide sequence against a protein sequence database;

(4) TBLASTN compares a query protein sequence against a nucleotide sequence database translated in all six reading frames; and

(5) TBLASTX compares the six-frame translations of a nucleotide query sequence against the six-frame translations of a nucleotide sequence database.

The term “cDNA” used herein denotes nucleic acids that synthesized from a mRNA template using reverse transcriptase.

The term “cDNA library” used herein denotes a library composed of complementary DNAs which are reverse-transcribed from mRNAs.

The term “complement” used herein denotes a polynucleotide sequence capable of forming base pairing with another polynucleotide sequence. For example, the sequence 5′-ATGGACTTACT-3′ binds to the complementary sequence 5′-AGTAAGTCCAT-3′.

The term “deletion” used herein denotes a removal of a portion of one or more amino acid residues/nucleotides from a gene.

The term “expressed sequence tags (ESTs)” used herein denotes short (200 to 500 base pairs) nucleotide sequence that derives from either 5′ or 3′ end of a cDNA.

The term “expression vector” used herein denotes nucleic acid constructs which contain a cloning site for introducing the DNA into vector, one or more selectable markers for selecting vectors containing the DNA, an origin of replication for replicating the vector whenever the host cell divides, a terminator sequence, a polyadenylation signal, and a suitable control sequence which can effectively express the DNA in a suitable host. The suitable control sequence may include promoter, enhancer and other regulatory sequences necessary for directing polymerases to transcribe the DNA.

The term “host cell” used herein denotes a cell which is used to receive, maintain, and allow the reproduction of an expression vector comprising DNA. Host cells are transformed or transfected with suitable vectors constructed using recombinant DNA methods. The recombinant DNA introduced with the vector is replicated whenever the cell divides.

The term “insertion” or “addition” used herein denotes the addition of a portion of one or more amino acid residues/nucleotides to a gene.

The term “in silico” used herein denotes a process of using computational methods (e.g., BLAST) to analyze DNA sequences.

The term “polymerase chain reaction (PCR)” used herein denotes a method which increases the copy number of a nucleic acid sequence using a DNA polymerase and a set of primers (about 20bp oligonucleotides complementary to each strand of DNA) under suitable conditions (successive rounds of primer annealing, strand elongation, and dissociation).

The term “protein” or “polypeptide” used herein denotes a sequence of amino acids in a specific order that can be encoded by a gene or by a recombinant DNA. It can also be chemically synthesized.

The term “nucleic acid sequence” or “polynucleotide” used herein denotes a sequence of nucleotide (guanine, cytosine, thymine or adenine) in a specific order that can be a natural or synthesized fragment of DNA or RNA. It may be single-stranded or double-stranded.

The term “reverse transcriptase-polymerase chain reaction (RT-PCR)” used herein denotes a process which transcribes mRNA to complementary DNA strand using reverse transcriptase followed by polymerase chain reaction to amplify the specific fragment of DNA sequences.

The term “transformation” used herein denotes a process describing the uptake, incorporation, and expression of exogenous DNA by prokaryotic host cells.

The term “transfection” used herein a process describing the uptake, incorporation, and expression of exogenous DNA by eukaryotic host cells.

The term “variant” used herein denotes a fragment of sequence (nucleotide or amino acid) inserted or deleted by one or more nucleotides/amino acids.

The present invention in the fist aspect provides the polypeptides of a novel human NjmuR1-related gene variant and the fragments thereof, as well as the nucleic acid sequences encoding the same.

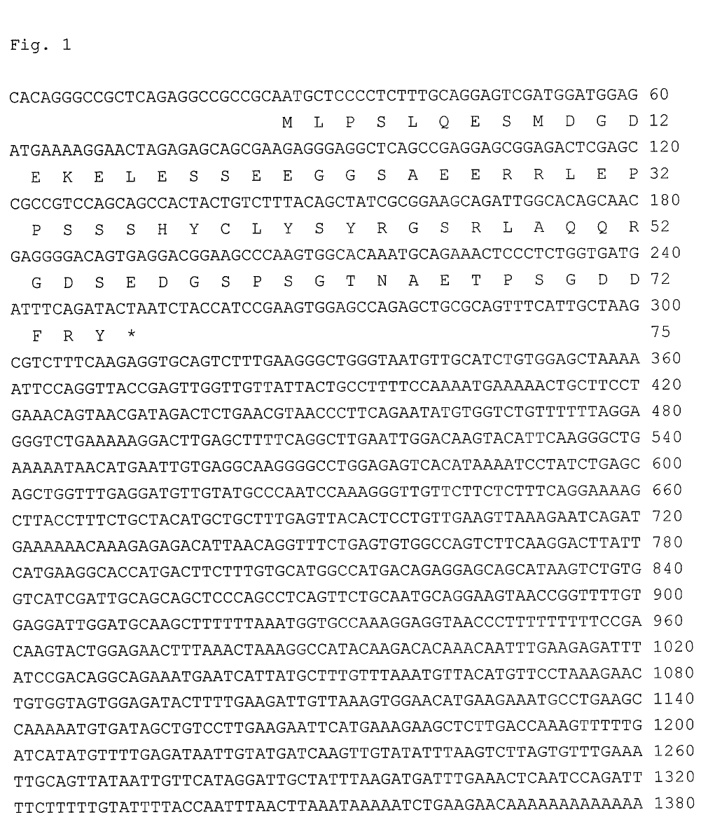

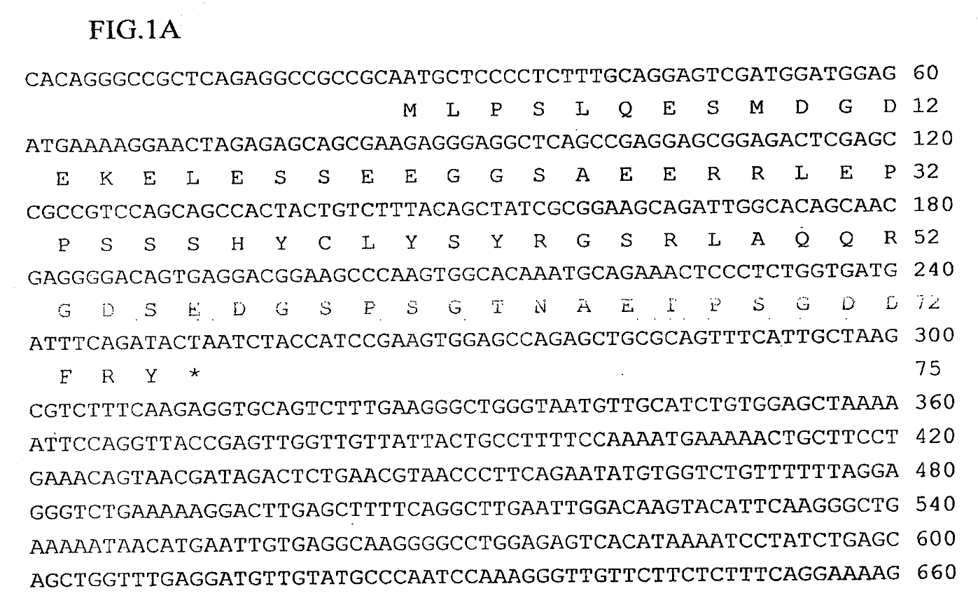

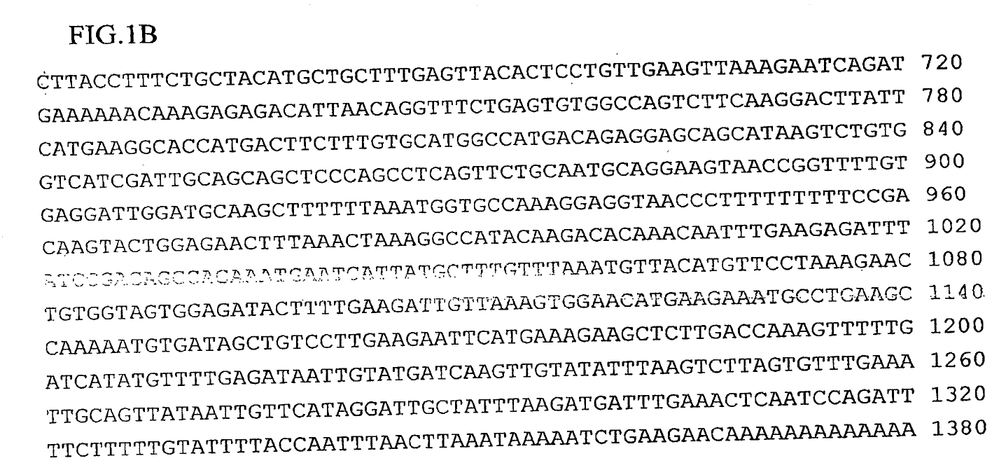

According to the present invention, human NjmuR1cDNA sequence was used to query the human lung EST databases (a normal lung, a large cell lung cancer, a squamous cell lung cancer and a small cell lung cancer) using BLAST program to search for NjmuR1-related gene variants. One human cDNA partial sequences (i.e., EST) showing similarity to NjmuR1 was identified from the ESTs deposited in the SCLC database. The cDNA clone, named NjmuR1V (NjmuR1variant), was then isolated from the SCLC cDNA library and sequenced. FIG. 1 shows the nucleic acid sequences of NjmuR1V (SEQ ID NOs: 1) and its corresponding amino acid sequences encoded thereby (SEQ ID NOs: 2).

The full-length of the NjmuR1V cDNA is a 1380 bp clone containing a 225 bp open reading frame (ORF) extending from 27 bp to 251 bp, which corresponds to an encoded protein of 75 amino acid residues with a predicted molecular mass of 8.3 kDa. The sequence around the initiation ATG codon of NjmuR1V (located at nucleotide 27 to 29 bp) was similar to the Kozak consensus sequence (A/GCCATGG) (Kozak, (1987) Nucleic Acids Res. 15: 8125-48; Kozak, (1991) J Cell Biol. 115: 887-903.).

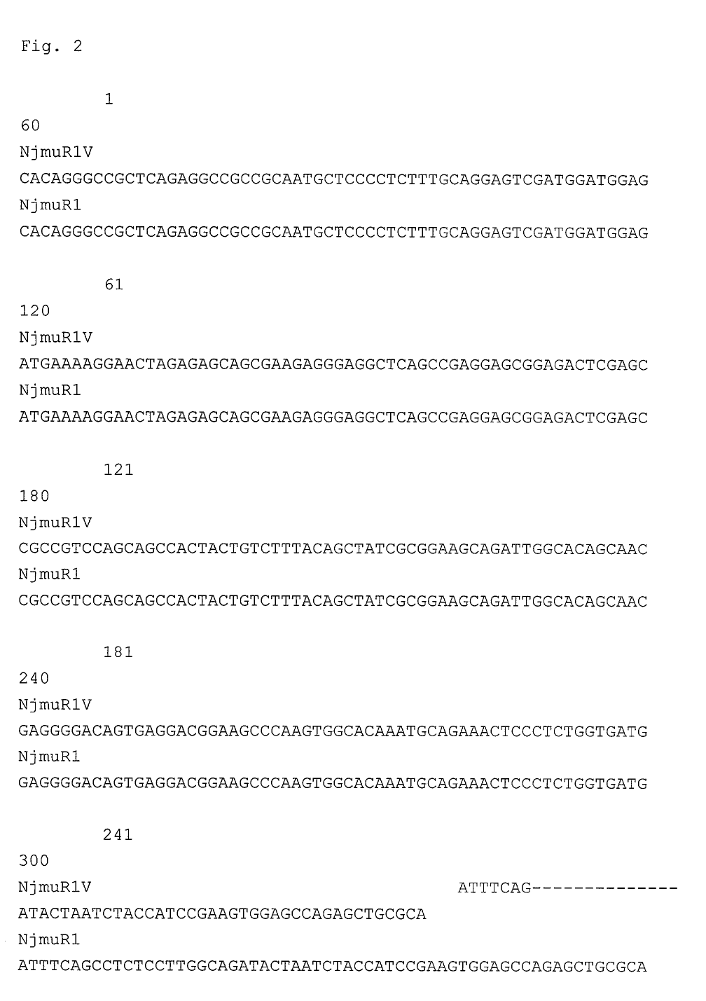

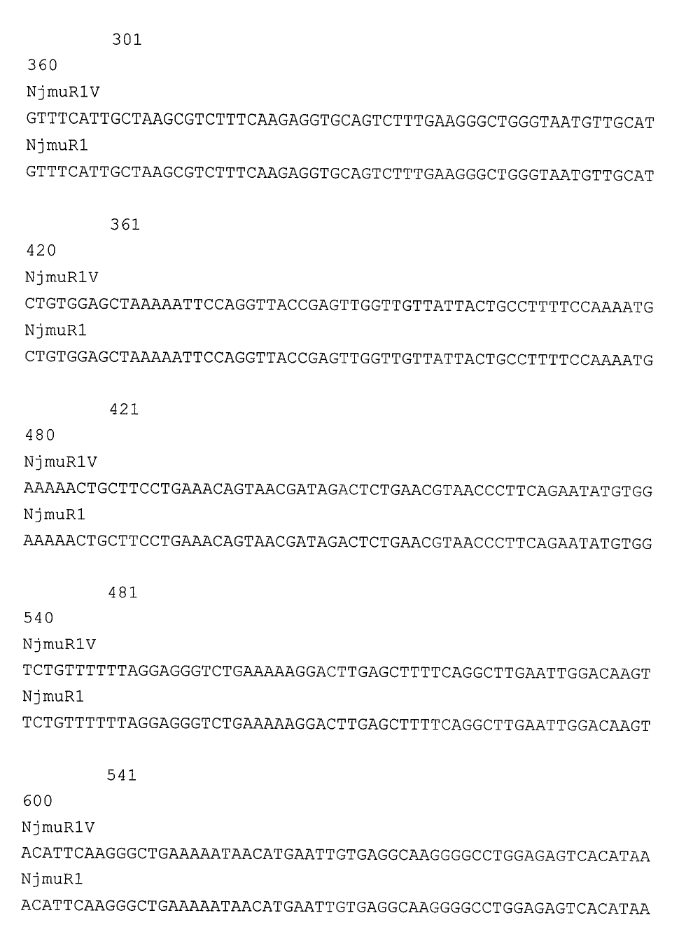

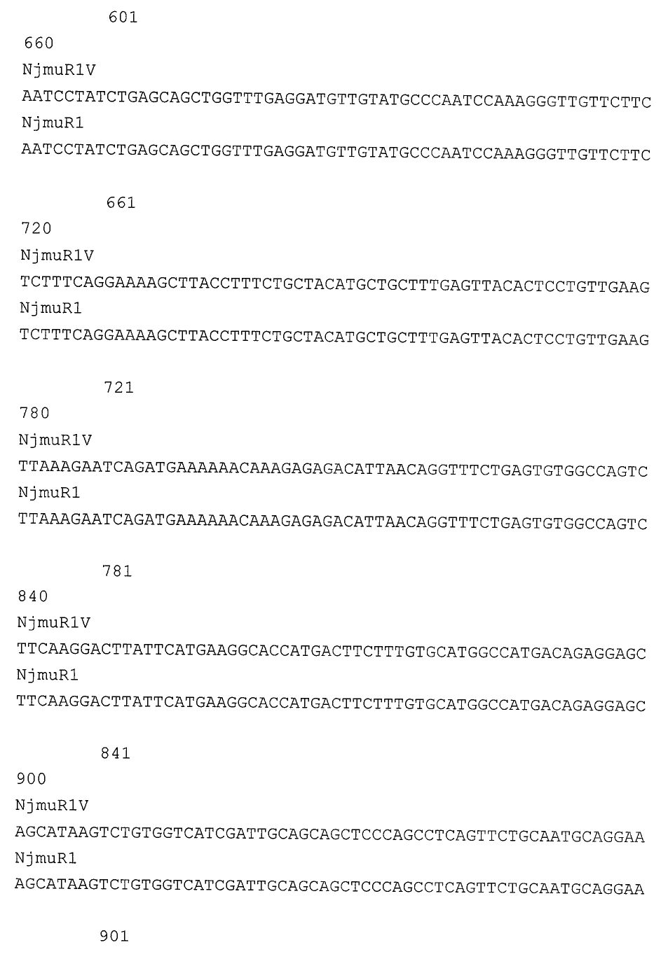

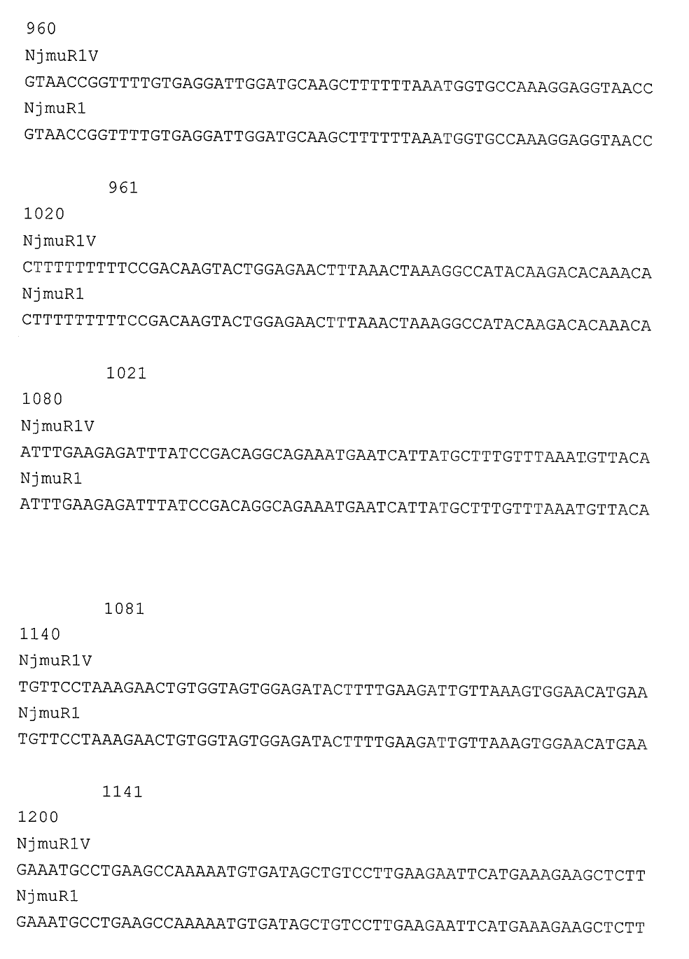

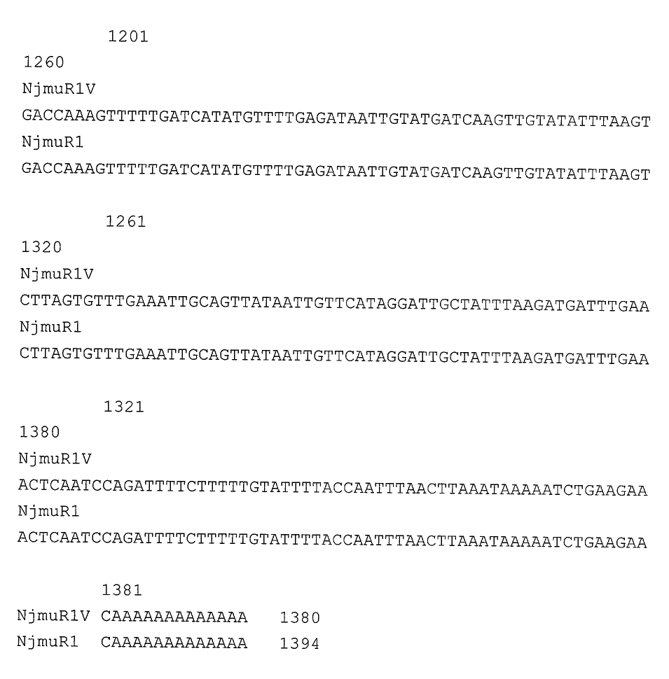

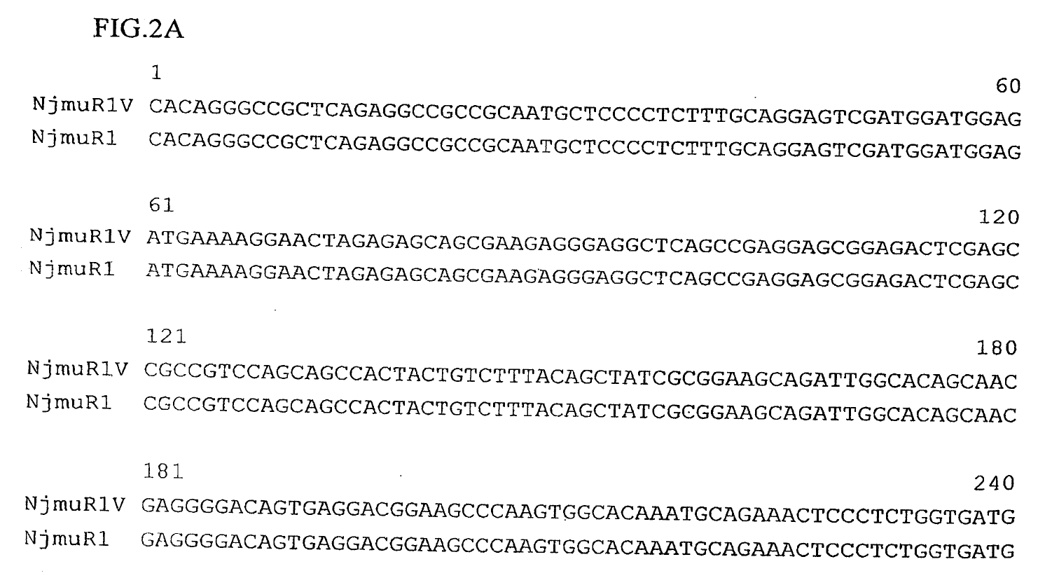

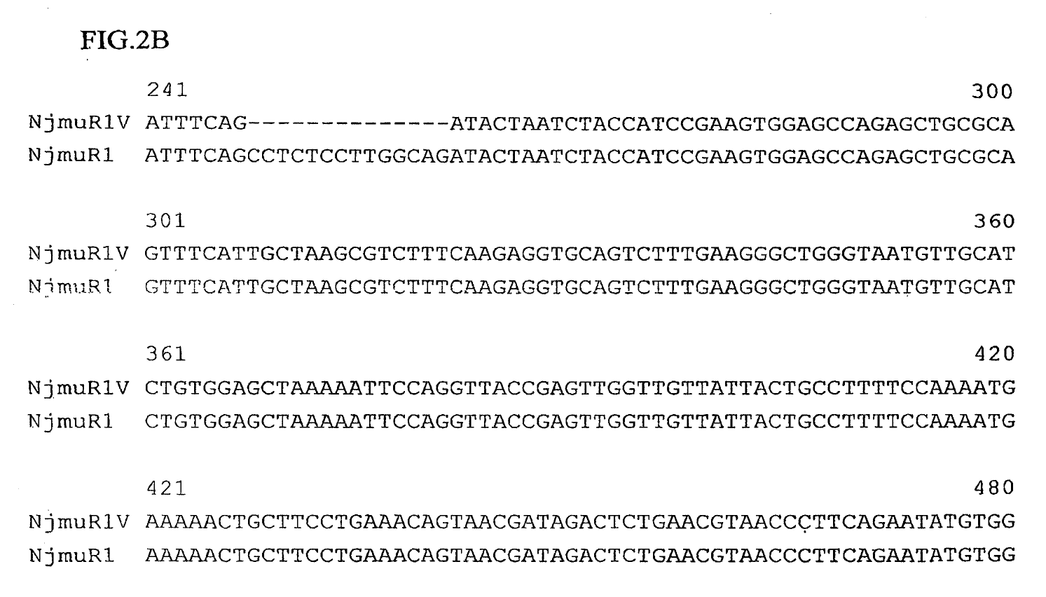

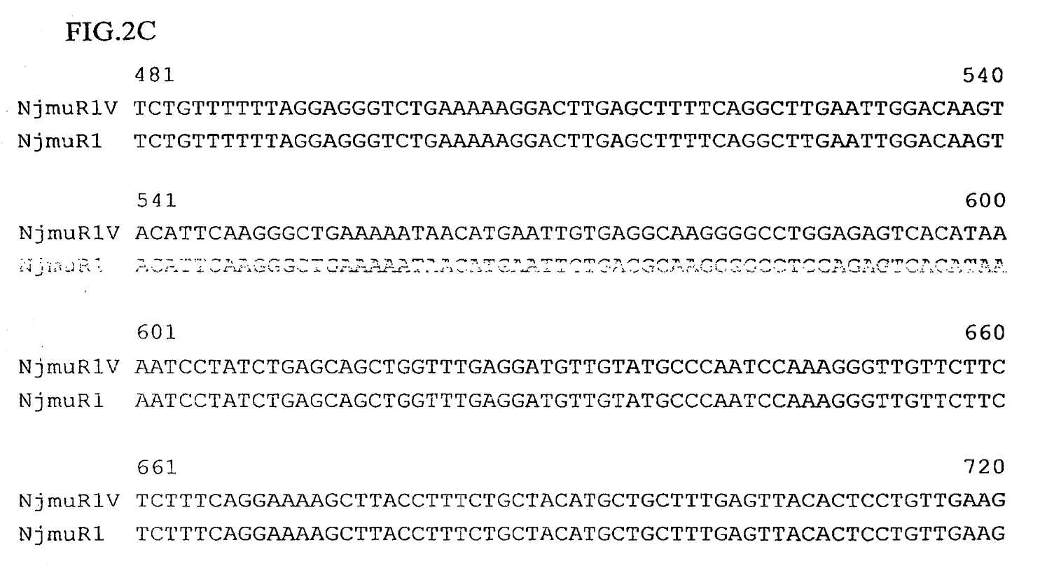

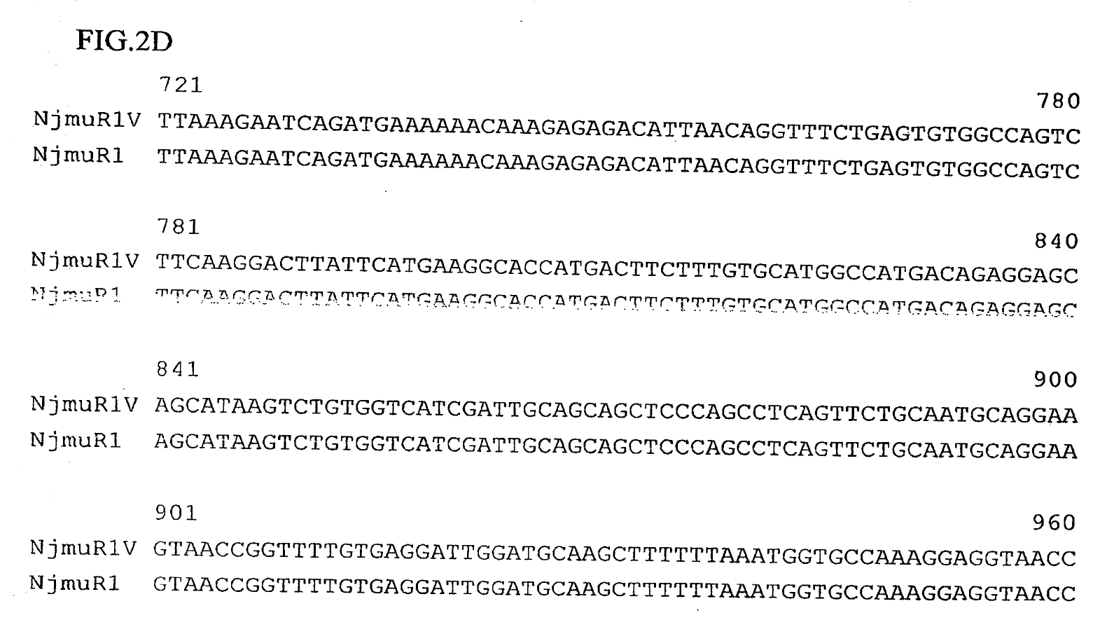

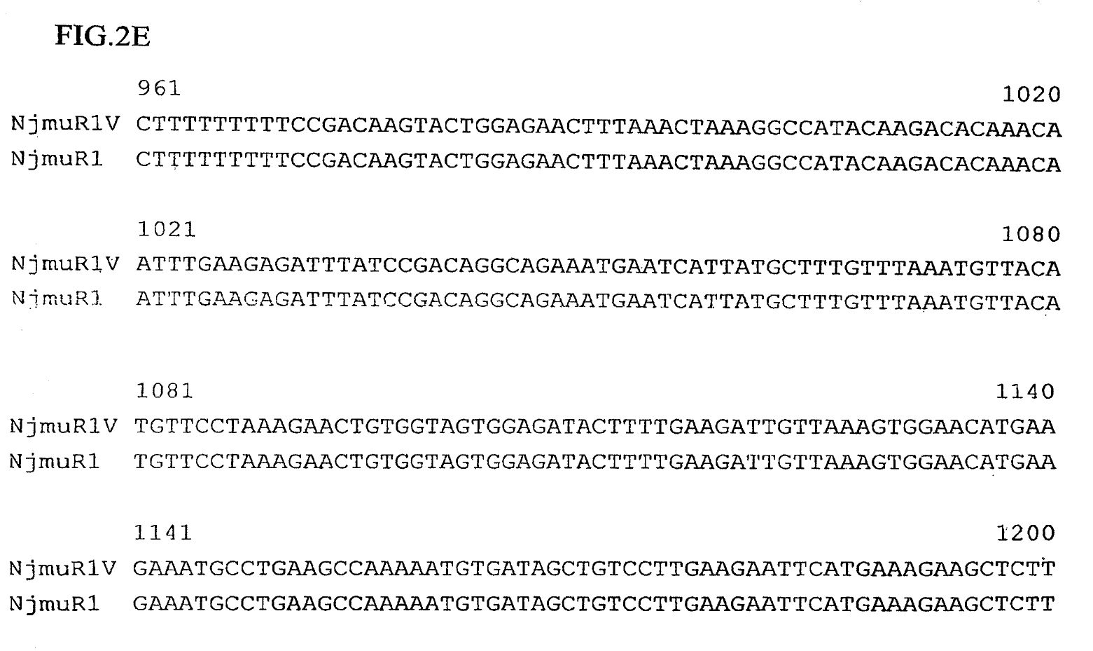

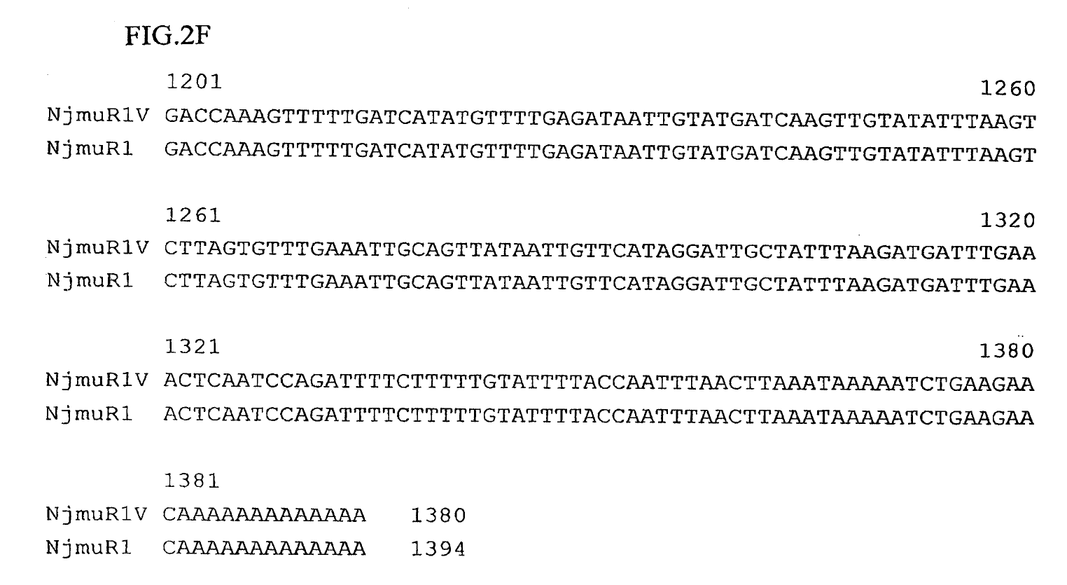

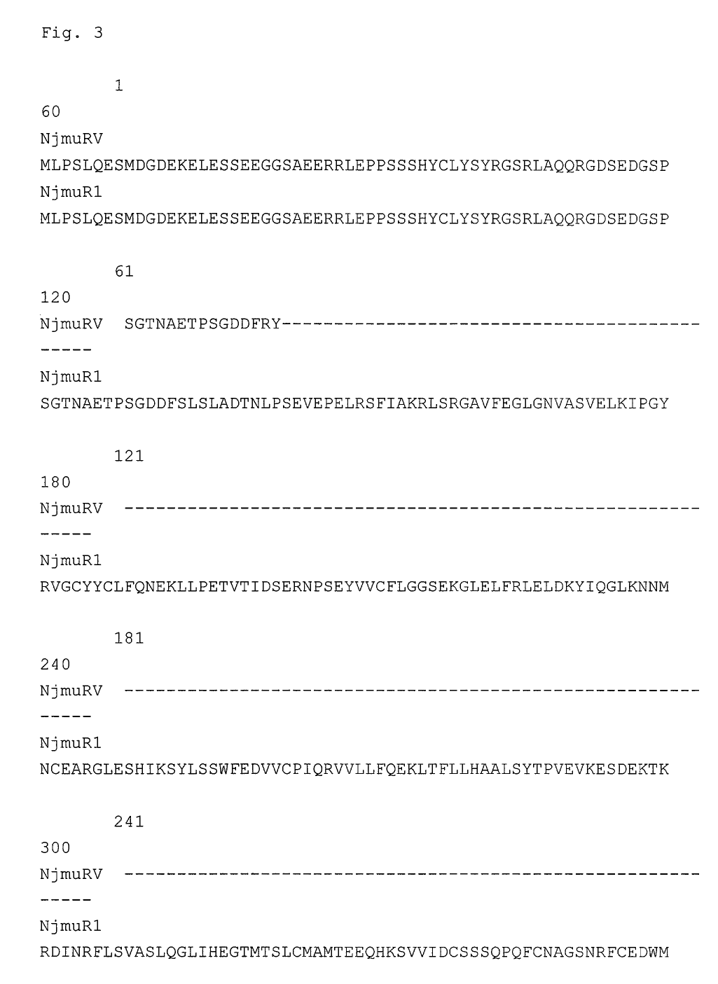

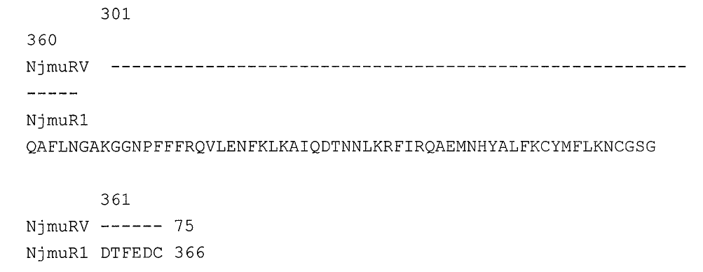

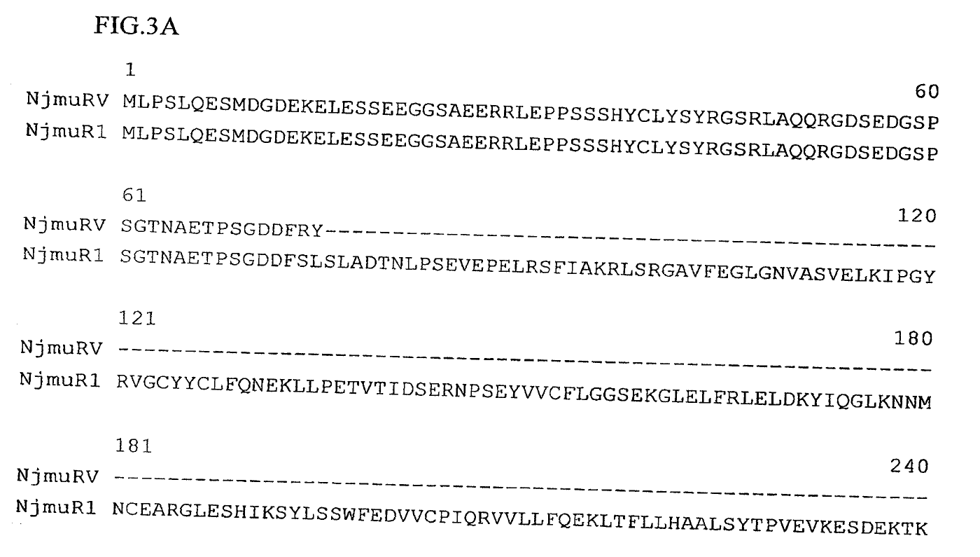

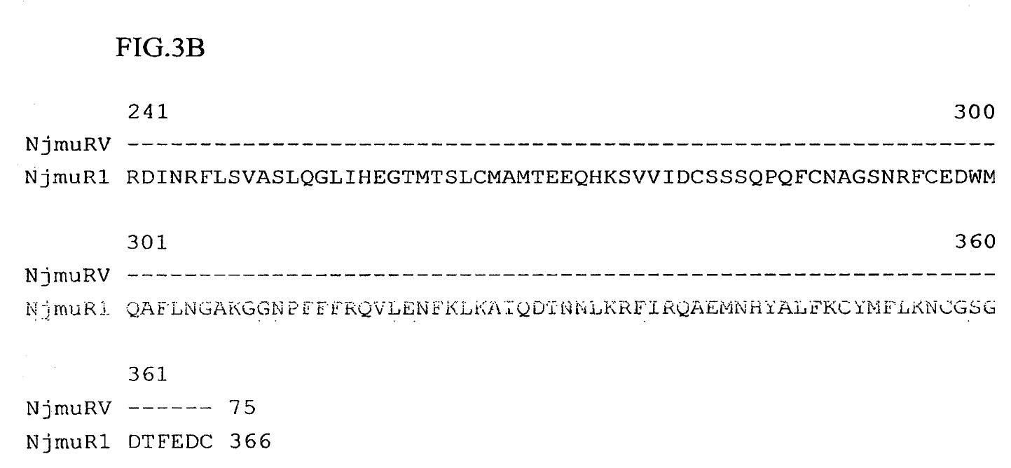

To determine the variation in sequence of NjmuR1V cDNA clone, an alignment of NjmuR1nucleotide/amino acid sequence with NjmuR1V was performed (FIGS. 2 and 3). One major genetic deletion was found in the aligned sequences, showing that NjmuR1V is a 14 bp deletion in the sequence of NjmuR1from 248-261 bp. The lacking of 14 bp causes a frame-shift in the amino acid sequence and generates a premature stop codon downstream the amino acid position 75 of NjmuR1V. Thus, the predicted amino acid sequence indicated that NjmuR1V is a C-terminal truncated protein of NjmuR1(FIG. 3).

In the present invention, a search of ESTs deposited in dbEST (Boguski et al. (1993) Nat Genet. 4: 332-3) at National Center of Biotechnology Information (NCBI) was performed to determine the tissue distribution of NjmuR1V in silico. The result of in silico Northern analysis showed that one EST (GenBank Accession Number BE266920) was found to confirm the absence of 14 bp region on NjmuR1V nucleotide sequence. This EST was also generated from a SCLC cDNA library, suggesting that the absence of 14 bp nucleotide fragment located between nucleotides 247 to 248 of NjmuR1V may serve as a useful marker for diagnosing diseases associated with the deficiency of NjmuR1gene, in particular, lung cancers, e.g. SCLC. Therefore, any nucleotide fragments comprising nucleotides 247 to 248 of NjmuR1V may be used as probes for determining the presence of NjmuR1V under highly stringent conditions. An alternative approach is that any set of primers for amplifying the fragment containing nucleotides 247 to 248 of NjmuR1V may be used for determining the presence of the variant.

According to the present invention, the polypeptide of the human RjmuR1V and the fragments thereof may be produced via genetic engineering techniques. For instance, they may be produced by appropriate host cells which have been transformed by DNAs that code for the desired polypeptide and the fragments thereof. The nucleotide sequence encoding the polypeptide of the RjmuR1Vor the fragments thereof is inserted into an appropriate expression vector, i.e., a vector which contains the necessary elements for the transcription and translation of the inserted coding sequence in a suitable host. The nucleic acid sequence is inserted into the vector in a manner such that it will be expressed under appropriate conditions (e.g., in proper orientation and correct reading frame and with appropriate expression sequences, including an RNA polymerase binding sequence and a ribosomal binding sequence).

Any method that is known to those skilled in the art may be used to construct expression vectors containing sequences encoding the polypeptide of the human RjmuR1V and appropriate transcriptional/translational control elements. These methods may include in vitro recombinant DNA and synthetic techniques, and in vivo genetic recombinants. (See, e.g., Sambrook, J. Cold Spring Harbor Press, Plainview N.Y., Ch. 4, 8, and 16-17; Ausubel, R. M. et al. (1995) Current protocols in Molecular Biology, John Wiley & Sons, New York N.Y., Ch. 9, 13, and 16.) A variety of expression vector/host systems may be utilized to express the polypeptide-coding sequence. These include, but not limited to, microorganisms such as bacteria transformed with recombinant bacteriophage, plasmid, or cosmid DNA expression vector; yeast transformed with yeast expression vector; insect cell systems infected with virus (e.g., baculovirus); plant cell system transformed with viral expression vector (e.g., cauliflower mosaic virus, CaMV, or tobacco mosaic virus, TMV); or animal cell system infected with virus (e.g., vaccina virus, adenovirus, etc.). Preferably, the host cell is a bacterium, and most preferably, the bacterium is E. coli.

Alternatively, the polypeptide of the human RjmuR1V or the fragments thereof may be synthesized by using chemical methods. For example, peptide synthesis can be performed using various solid-phase techniques (Roberge, J. Y. et al. (1995) Science 269: 202 to 204). Automated synthesis may be achieved by using the ABI 431A peptide synthesizer (Perkin-Elmer).

According to the present invention, the fragments of the polypeptides and nucleic acid sequences of the human NjmuR1V may be used as immunogens and primers or probes, respectively. Preferably, the purified fragments of the human NjmuR1V are used. The fragments may be produced by enzymatic digestion, chemical cleavage of isolated or purified polypeptide or nucleic acid sequences, or chemical synthesis and then may be isolated or purified. Such isolated or purified fragments of the polypeptides and nucleic acid sequences can be directly used as immunogens and primers or probes, respectively.

The present invention further provides the antibodies which specifically bind one or more out-surface epitopes of the polypeptide of the human NjmuR1V.

According to the present invention, immunization of mammals with immunogens described herein, preferably humans, rabbits, rats, mice, sheep, goats, cows, or horses, is performed following procedures well known to those skilled in the art, for the purpose of obtaining antisera containing polyclonal antibodies or hybridoma lines secreting monoclonal antibodies.

Monoclonal antibodies can be prepared by standard techniques, given the teachings contained herein. Such techniques are disclosed, for example, in U.S. Pat. No. 4,271,145 and U.S. Pat. No. 4,196,265. Briefly, an animal is immunized with the immunogen. Hybridomas are prepared by fusing spleen cells from the immunized animal with myeloma cells. The fusion products are screened for those producing antibodies that bind to the immunogen. The positive hybridoma clones are isolated, and the monoclonal antibodies are recovered from those clones.

Immunization regimens for production of both polyclonal and monoclonal antibodies are well-known in the art. The immunogen may be injected by any of a number of routes, including subcutaneous, intravenous, intraperitoneal, intradermal, intramuscular, mucosal, or a combination thereof. The immunogen may be injected in soluble form, aggregate form, attached to a physical carrier, or mixed with an adjuvant, using methods and materials well-known in the art. The antisera and antibodies may be purified using column chromatography methods well known to those skilled in the art.

According to the present invention, antibody fragments which contain specific binding sites for the polypeptides or fragments thereof may also be generated. For example, such fragments include, but are not limited to, F(ab′)₂ fragments produced by pepsin digestion of the antibody molecule and Fab fragments generated by reducing the disulfide bridges of the F(ab′)₂ fragments.

Many gene variants have been found to be associated with diseases (Stallings-Mann et al., (1996) Proc Natl Acad Sci U S A 93: 12394-9; Liu et al., (1997) Nat Genet 16:328-9; Siffert et al., (1998) Nat Genet 18: 45 to 8; Lukas et al., (2001) Cancer Res 61: 3212 to 9). Since NjmuR1V clone was isolated from SCLC cDNA library and its expression in SCLC was confirmed by in silico Northern analysis, it is advisable that NjmuR1V may serve as markers for the diagnosis of diseases associated with the deficiency of NjmuR1gene, e.g. lung cancers, more specifically SCLC. Thus, the expression level of NjmuR1V relative to NjmuR1may be a useful indicator for screening of patients suspected of having such diseases, and the index of relative expression level (mRNA or protein) may confer an increased susceptibility to such diseases.

Accordingly, the subject invention also provides methods for diagnosing diseases associated with the deficiency of NjmuR1gene in a mammal, in particular, lung cancers, e.g. SCLC.

The method for diagnosing the diseases associated with the deficiency of NjmuR1gene may be performed by detecting the nucleotide sequence of the human NjmuR1V of the invention which comprises the steps of: (1) extracting total RNA of cells obtained from a mammal; (2) amplifying the RNA by reverse transcriptase-polymerase chain reaction (RT-PCR) with a set of primers to obtain a cDNA comprising the fragments comprising nucleotides 246 to 251 of SEQ ID NO: 1; and (3) detecting whether the cDNA sample is obtained. If necessary, the amount of the obtained cDNA sample may be detected.

In the above embodiment, one of the primers may be designed to have a sequence comprising the nucleotides of SEQ ID NO: 1 containing nucleotides 246 to 251, and the other may be designed to have a sequence complementary to the nucleotides of SEQ ID NO: 1 at any other locations downstream of nucleotide 251. Alternatively, one of the primers may be designed to have a sequence complementary to the nucleotides of SEQ ID NO: 1 containing nucleotides 246 to 251, and the other may be designed to have a sequence comprising the nucleotides of SEQ ID NO: 1 at any other locations upstream of nucleotide 246. In this case, only NjmuR1V will be amplified.

Alternatively, one of the primers may be designed to have a sequence comprising the nucleotides of SEQ ID NO: 1 upstream of nucleotide 247 and the other may be designed to have a sequence complementary to the nucleotides of SEQ ID NO: 1 downstream of nucleotide 248. Alternatively, one of the primers may be designed to have a sequence complementary to the nucleotides of SEQ ID NO: 1 upstream of nucleotide 247 and the other may be designed to have a sequence comprising the nucleotides of SEQ ID NO: 1 downstream of nucleotide 248. In this case, both NjmuR1and NjmuR1V will be amplified. The length of the PCR fragment from NjmuR1V will be 14 bp shorter than that from NjmuR1.

Preferably, the primer of the invention contains 15 to 30 nucleotides.

Total RNA may be isolated from patient samples by using TRIZOL reagents (Life Technology). Tissue samples (e.g., biopsy samples) are powdered under liquid nitrogen before homogenization. RNA purity and integrity are assessed by absorbance at 260/280 nm and by agarose gel electrophoresis. The set of primers designed to amplify the expected size of specific PCR fragments of RjmuR1V can be used. PCR fragments are analyzed on a 1% agarose gel using five microliters (10%) of the amplified products. To determine the expression level of the gene variant, the intensity of the PCR products may be determined by using the Molecular Analyst program (version 1.4.1; Bio-Rad).

The RT-PCR experiment may be performed according to the manufacturer's instructions (Boehringer Mannheim). A 50 μl reaction mixture containing 2 μl total RNA (0.1 μg/μl), 1 μl each primer (20 pM), 1 μl each dNTP (10 mM), 2.5 μl DTT solution (100 mM), 10 μl 5× RT-PCR buffer, 1 μl enzyme mixture, and 28.5 μl sterile distilled water may be subjected to the conditions such as reverse transcription at 60° C. for 30 minutes followed by 35 cycles of denaturation at 94° C. for 2 minutes, annealing at 60° C. for 2 minutes, and extension at 68° C. for 2 minutes. The RT-PCR analysis may be repeated twice to ensure reproducibility, for a total of three independent experiments.

Another embodiment for diagnosing the diseases associated with the deficiency of RjmuR1 gene may be performed by detecting the nucleotide sequences of the human RjmuR1V of the invention which comprises the steps of: (1) extracting total RNA from a sample obtained from the mammal; (2) amplifying the RNA by reverse transcriptase-polymerase chain reaction (RT-PCR) to obtain a cDNA sample; (3) bringing the cDNA sample into contact with the nucleic acid of SEQ ID NO: 1 and the fragments thereof; and (4) detecting whether the cDNA sample hybridizes with the nucleic acid of SEQ ID NO: 1 or the fragments thereof. If necessary, the amount of hybridized sample may be detected.

The expression of gene variants can also be analyzed using Northern Blot hybridization approach. Specific fragment comprising nucleotides 246 to 251 of NjmuR1V may be amplified by polymerase chain reaction (PCR) using primer set designed for RT-PCR. The amplified PCR fragment may be labeled and serve as a probe to hybridize the membranes containing total RNAs extracted from the samples under the conditions of 55° C. in a suitable hybridization solution for 3 hr. Blots may be washed twice in 2×SSC, 0.1% SDS at room temperature for 15 minutes each, followed by two washes in 0.1×SSC and 0.1% SDS at 65° C. for 20 minutes each. After these washes, blot may be rinsed briefly in suitable washing buffer and incubated in blocking solution for 30 minutes, and then incubated in suitable antibody solution for 30 minutes. Blots may be washed in washing buffer for 30 minutes and equilibrated in suitable detection buffer before detecting the signals. Alternatively, the presence of gene variants (cDNAs or PCR) can be detected using microarray approach. The cDNAs or PCR products corresponding to the nucleotide sequences of the present invention may be immobilized on a suitable substrate such as a glass slide. Hybridization can be preformed using the labeled mRNAs extracted from samples. After hybridization, nonhybridized mRNAs are removed. The relative abundance of each labeled transcript, hybridizing to a cDNA/PCR product immobilized on the microarray, can be determined by analyzing the scanned images.

According to the present invention, the method for diagnosing the diseases associated with the deficiency of RjmuR1 gene may also be performed by detecting the polypeptide encoded by the RjmuR1V of the invention. For instance, the polypeptide in protein samples obtain from the mammal may be determined by, but not limited to, the immunoassay wherein the antibody specifically binding to the polypeptide of the invention is contacted with the protein samples, and the antibody-polypeptide complex is detected. If necessary, the amount of the anti-body-polypeptide complex can be determined.

The polypeptides of RjmuR1V may be expressed in prokaryotic cells by using suitable prokaryotic expression vectors. The cDNA fragments of NjmuR1V gene encoding the amino acid coding sequence may be PCR amplified using primer set with restriction enzyme digestion sites incorporated in the 5′ and 3′ ends, respectively. The PCR products can then be enzyme digested, purified, and inserted into the corresponding sites of prokaryotic expression vector in-frame to generate recombinant plasmids. Sequence fidelity of this recombinant DNA can be verified by sequencing. The prokaryotic recombinant plasmids may be transformed into host cells (e.g., E. coli BL21 (DE3)). Recombinant protein synthesis may be stimulated by the addition of 0.4 mM isopropylthiogalactoside (IPTG) for 3 h. The bacterially-expressed proteins may be purified.

The polypeptide of the gene variant may be expressed in animal cells by using eukaryotic expression vectors. Cells may be maintained in Dulbecco's modified Eagle's medium (DMEM) supplemented with 10% fetal bovine serum (FBS; Gibco BRL) at 37° C. in a humidified 5% CO₂ atmosphere. Before transfection, the nucleotide sequence of each of the gene variant may be amplified with PCR primers containing restriction enzyme digestion sites and ligated into the corresponding sites of eukaryotic expression vector in-frame. Sequence fidelity of this recombinant DNA can be verified by sequencing. The cells may be plated in 12-well plates one day before transfection at a density of 5×10⁴ cells per well. Transfections may be carried out using Lipofectaminutese Plus transfection reagent according to the manufacturer's instructions (Gibco BRL). Three hours following transfection, medium containing the complexes may be replaced with fresh medium. Forty-eight hours after incubation, the cells may be scraped into lysis buffer (0.1 M Tris HCl, pH 8.0, 0.1% Triton X-100) for purification of expressed proteins. After these proteins are purified, monoclonal antibodies against these purified proteins (NjmuR1V) may be generated using hybridoma technique according to the conventional methods (de StGroth and Scheidegger, (1980) J Immunol Methods 35:1-21; Cote et al. (1983) Proc Natl Acad Sci U S A 80: 2026-30; and Kozbor et al. (1985) J Immunol Methods 81:31-42).

According to the present invention, the presence of the polypeptides of the gene variant in samples obtained from the mammal suspected of having diseases associated with the deficiency of NjmuR1gene may be determined by, but not limited to, Western blot analysis. Proteins extracted from samples may be separated by SDS-PAGE and transferred to suitable membranes such as polyvinylidene difluoride (PVDF) in transfer buffer (25 mM Tris-HCl, pH 8.3, 192 mM glycine, 20% methanol) with a Trans-Blot apparatus for 1 h at 100 V (e.g., Bio-Rad). The proteins can be immunoblotted with specific antibodies. For example, membrane blotted with extracted proteins may be blocked with suitable buffers such as 3% solution of BSA or 3% solution of nonfat milk powder in TBST buffer (10 mM Tris-HCl, pH 8.0, 150 mM NaCl, 0.1% Tween 20) and incubated with monoclonal antibody directed against the polypeptides of gene variants. Unbound antibody is removed by washing with TBST for 5×1 minutes. Bound antibody may be detected using commercial ECL Western blotting detecting reagents.

The following examples are provided for illustration, but not for limiting the invention.

### EXAMPLES

Analysis of Human Lung EST Databases

Expressed sequence tags (ESTs) generated from the large-scale PCR-based sequencing of the 5′-end of human lung (normal, SCLC, squamous cell lung cancer and large cell lung cancer) cDNA clones were compiled and served as EST databases. Sequence comparisons against the nonredundant nucleotide and protein databases were performed using BLASTN and BLASTX programs (Altschul et al., (1997) Nucleic Acids Res. 25: 3389-3402; Gish and States, (1993) Nat Genet 3:266-272), at the National Center for Biotechnology Information (NCBI) with a significance cutoff of p<10⁻¹⁰. ESTs representing putative NjmuR1V gene were identified during the course of EST generation.

Isolation of cDNA Clones

One cDNA clone exhibiting EST sequence similar to the NjmuR1 gene was isolated from the SCLC cDNA library and named NjmuR1V. The inserts of these clones were subsequently excised in vivo from the λKZAP Express vector using the ExAssist/XLOLR helper phage system (Stratagene). Phagemid particles were excised by coinfecting XL1-BLUE dMRF' cells with ExAssist helper phage. The excised pBluescript phagemids were used to infect E. coli XLOLR cells, which lack the amber suppressor necessary for ExAssist phage replication. Infected XLOLR cells were selected using kanamycin resistance. Resultant colonies contained the double stranded phagemid vector with the cloned cDNA insert. A single colony was grown overnight in LB-kanamycin, and DNA was purified using a Qiagen plasmid purification kit.

Full Length Nucleotide Sequencing and Database Comparisons

Phagemid DNA was sequenced using the Epicentre#SE910LC SequiTherm EXCEL™II DNA Sequencing Kit for 4200S-2 Global NEW IR² DNA sequencing system (LI-COR). Using the primer-walking approach, full-length sequence was determined. Nucleotide and protein searches were performed using BLAST against the non-redundant database of NCBI.

In Silico Tissue Distribution (Northern) Analysis

The coding sequence for each cDNA clones was searched against the dbEST sequence database (Boguski et al., (1993) Nat Genet. 4: 332-3) using the BLAST algorithm at the NCBI website. ESTs derived from each tissue were used as a source of information for transcript tissue expression analysis. Tissue distribution for each isolated cDNA clone was determined by ESTs matching to that particular sequence variants (insertions or deletions) with a significance cutoff of p<10⁻¹⁰.

References

Abujiang et al. Loss of heterozygosity (LOH) at 17 q and 14 q in human lung cancers. Oncogene, 17:3029-33, (1998).

Altschul et al., Gapped BLAST and PSI-BLAST: a new generation of protein database search programs, Nucleic Acids Res, 25: 3389-3402, (1997).

Ausubel et al., Current protocols in Molecular Biology, John Wiley & Sons, New York N.Y., ch. 9, 13, and 16, (1995).

Boguski et al., dbEST—database for “expressed sequence tags”. Nat Genet. 4: 332-3, (1993).

Carney, The biology of lung cancer. Curr. Opin. Oncol. 4: 292-8, (1992a).

Carney, Biology of small-cell lung cancer. Lancet 339: 843-6, (1992b).

Cote et al., Generation of human monoclonal antibodies reactive with cellular antigens, Proc Natl Acad Sci U S A 80: 2026-30 (1983).

de StGroth and Scheidegger, Production of monoclonal antibodies: strategy and tactics, J Immunol Methods 35:1-21, (1980).

Gish and States, Identification of protein coding regions by database similarity search, Nat Genet, 3:266-272, (1993).

Ihde and Minna, Non-small cell lung cancer. Part II: Treatment. Curr. Probl. Cancer 15: 105-54, (1991).

Kato et al. Establishment of a human small cell lung carcinoma cell line carrying amplification of c-myc gene and chromosomal translocation of t(3 p;6 p) and t(12 q;17 p). Jpn J Cancer Res, 84:355-9, (1993).

Konishi et al. Detailed deletion mapping suggests the involvement of a tumor suppressor gene at 17p 13.3, distal to p53, in the pathogenesis of lung cancers. Oncogene, 17:2095-100, (1998).

Kozak, An analysis of 5′-noncoding sequences from 699 vertebrate messenger RNAs. Nucleic Acids Res, 15: 8125-48, (1987).

Kozak, An analysis of vertebrate mRNA sequences: intimations of translational control, J Cell Biol, 115: 887-903, (1991).

Kozbor et al., Specific immunoglobulin production and enhanced tumorigenicity following as cites growth of human hybridomas, J Immunol Methods, 81:31-42 (1985).

Levin et al. Identification of novel regions of altered DNA copy number in small cell lung tumors. Genes Chromosomes Cancer, 13:175-85, (1995).

Liu et al., Silent mutation induces exon skipping of fibrillin-1 gene in Marfan syndrome. Nat Genet 16:328-9, (1997).

Lukas et al., Alternative and aberrant messenger RNA splicing of the mdm2 oncogene in invasive breast cancer. Cancer Res 61:3212-9, (2001).

Roberge et al., A strategy for a convergent synthesis of N-linked glycopeptides on a solid support. Science 269:202-4, (1995).

Sambrook, J. Cold Spring Harbor Press, Plainview N.Y., ch. 4, 8, and 16-17.

Sameshima et al. Point mutation of the p53 gene resulting in splicing inhibition in small cell lung carcinoma. Biochem Biophys Res Commun, 173:697-703, (1990).

Sethi, Science, medicine, and the future. Lung cancer, BMJ, 314: 652-655, (1997).

Sha et al., GenBank Accession No. AF305686.

Shimizu et al. Loss of heterozygosity on chromosome arm 17 p in small cell lung carcinomas, but not in neurofibromas, in a patient with von Recklinghausen neurofibromatosis. Cancer, 71:725-8, (1993).

Siffert et al., Association of a human G-protein beta3 subunit variant with hypertension. Nat Genet, 18:45-8, (1998).

Smyth et al., The impact of chemotherapy on small cell carcinoma of the bronchus. Q J Med, 61: 969-76, (1986).

Stallings-Mann et al., Alternative splicing of exon 3 of the human growth hormone receptor is the result of an unusual genetic polymorphism. Proc Natl Acad Sci U S A 93:12394-9, (1996).

Strausberg, R. EST Accession No. BE266920.

What is claimed is:

1. An isolated polypeptide comprising the amino acid sequence of SEQ ID NO: 2, and fragments thereof.

2. An isolated nucleic acid encoding the polypeptide of claim 1, and fragments thereof.

3. The isolated nucleic acid of claim 2, which comprises the nucleotide sequence of SEQ ID NO: 1.

4. The isolated nucleic acid of claim 3, wherein the fragments comprise the nucleotides 246 to 251 of SEQ ID NO: 1.

5. An expression vector comprising the nucleic acid of any one of claims 2 to 4.

6. A host cell transformed with the expression vector of claim 5.

7. A method for producing the polypeptide of claim 1, which comprises the steps of:
(1) culturing the host cell of claim 6 under a condition suitable for the expression of the polypeptide; and
(2) recovering the polypeptide from the host cell culture.

8. An antibody specifically binding to the polypeptide of claim 1.

9. A method for diagnosing the diseases associated with the deficiency of human RjmuR1 gene in a mammal, in particular lung cancers, which comprises detecting the nucleic acid of any one of claims 2 to 4 or the polypeptide of claim 1.

10. The method of claim 9, wherein the disease is small cell lung cancer.

11. The method of claim 9, wherein the detection of the nucleic acid of any one claims 2 to 4 comprises the steps of:
(1) extracting total RNA from a sample obtained from the mammal;
(2) amplifying the RNA by reverse transcriptase-polymerase chain reaction (RT-PCR) with a pair of primers to obtain a cDNA sample comprising the nucleotides 246 to 251 of SEQ ID NO: 1; and
(3) detecting whether the cDNA sample is obtained.

12. The method of claim 11, wherein one of the primers has a sequence comprising the nucleotides of SEQ ID NO: 1 containing nucleotides 246 to 251, and the other has a sequence complementary to the nucleotides of SEQ ID NO: 1 at any other locations downstream of nucleotide 251, or one of the primers has a sequence complementary to the nucleotides of SEQ ID NO: 1 containing nucleotides 246 to 251, and the other has a sequence comprising the nucleotides of SEQ ID NO: 1 at any other locations upstream of nucleotide 246.

13. The method of claim 11, wherein one of the primers has a sequence comprising the nucleotides of SEQ ID NO: 1 upstream of nucleotide 247 and the other has a sequence complementary to the nucleotides of SEQ ID NO: 1 downstream of nucleotide 248, or one of the primers has a sequence complementary to the nucleotides of SEQ ID NO: 1 upstream of nucleotide 247 and the other has a sequence comprising the nucleotides of SEQ ID NO: 1 downstream of nucleotide 248.

14. The method of claim 13, wherein the cDNA sample amplified from SEQ ID NO: 1 is 14 bp shorter than the cDNA sample amplified from RjmuR1.

15. The method of claim 11 further comprising the step of detecting the amount of the amplified cDNA sample.

16. The method of claim 9, wherein the detection of the nucleic acid of any one of claims 2 to 4 comprises the steps of:
(1) extracting the total RNA of a sample obtained from the mammal;
(2) amplifying the RNA by reverse transcriptase-polymerase chain reaction (RT-PCR) to obtain a cDNA sample;
(3) bringing the cDNA sample into contact with the nucleic acid of any one of claims 2 to 4; and
(4) detecting whether the cDNA sample hybridizes with the nucleic acid of any one of claims 2 to 4.

17. The method of claim 16 further comprising the step of detecting the amount of hybridized sample.

18. The method of claim 9, wherein the detection of the polypeptide of claim 1 comprises the steps of contacting the antibody of claim 8 with a protein sample obtained from the mammal, and detecting whether an antibody-polypeptide complex is formed.

19. The method of claim 18 further comprising the step of detecting the amount of the antibody-polypeptide complex.


SEQUENCE LISTING (2 sequences)

SEQ ID NO: 1 - DNA, 1380 bp, Homo Sapien
cacagggccgctcagaggccgccgcaatgctcccctctttgcaggagtcgatggatggagatgaaaaggaactagagagcagcgaagagggaggctcagccgaggagcggagactcgagccgccgtccagcagccactactgtctttacagctatcgcggaagcagattggcacagcaacgaggggacagtgaggacggaagcccaagtggcacaaatgcagaaactccctctggtgatgatttcagatactaatctaccatccgaagtggagccagagctgcgcagtttcattgctaagcgtctttcaagaggtgcagtctttgaagggctgggtaatgttgcatctgtggagctaaaaattccaggttaccgagttggttgttattactgccttttccaaaatgaaaaactgcttcctgaaacagtaacgatagactctgaacgtaacccttcagaatatgtggtctgttttttaggagggtctgaaaaaggacttgagcttttcaggcttgaattggacaagtacattcaagggctgaaaaataacatgaattgtgaggcaaggggcctggagagtcacataaaatcctatctgagcagctggtttgaggatgttgtatgcccaatccaaagggttgttcttctctttcaggaaaagcttacctttctgctacatgctgctttgagttacactcctgttgaagttaaagaatcagatgaaaaaacaaagagagacattaacaggtttctgagtgtggccagtcttcaaggacttattcatgaaggcaccatgacttctttgtgcatggccatgacagaggagcagcataagtctgtggtcatcgattgcagcagctcccagcctcagttctgcaatgcaggaagtaaccggttttgtgaggattggatgcaagcttttttaaatggtgccaaaggaggtaaccctttttttttccgacaagtactggagaactttaaactaaaggccatacaagacacaaacaatttgaagagatttatccgacaggcagaaatgaatcattatgctttgtttaaatgttacatgttcctaaagaactgtggtagtggagatacttttgaagattgttaaagtggaacatgaagaaatgcctgaagccaaaaatgtgatagctgtccttgaagaattcatgaaagaagctcttgaccaaagtttttgatcatatgttttgagataattgtatgatcaagttgtatatttaagtcttagtgtttgaaattgcagttataattgttcataggattgctatttaagatgatttgaaactcaatccagattttctttttgtattttaccaatttaacttaaataaaaatctgaagaacaaaaaaaaaaaaa

SEQ ID NO: 2 - protein, 75 aa, Homo Sapien
MLPSLQESMDGDEKELESSEEGGSAEERRLEPPSSSHYCLYSYRGSRLAQQRGDSEDGSPSGTNAETPSGDDFRY